(19) United States
(12) Patent Application Publication
(10) Pub. No.: US 2006/0106440 A1
(43) Pub. Date: May 18, 2006

(54) Epidural electrode for use during spinal surgery

(75) Inventors: Amayur P. Chandran, Brownsville, TX (US); Rajendran K. Pisharody, Brownsville, TX (US); Madhavan Pisharodi, Brownsville, TX (US)

Correspondence Address: Mark R. Wisner;WISNER & ASSOCIATES, Suite 930, 2925 Briarpark, Houston, TX, 77042-3728, US

(73) Assignee: Perumala Corporation

(21) Appl. No.: 10/989,816

(22) Filed: Nov. 16, 2004

(51) Int. Cl. A61N 1/05

(52) U.S. Cl. 607/117

(57) ABSTRACT

An electrode for use in monitoring MEPs during spinal instrumentation. A thin, flexible strip of insulative material is provided with conductive tracks on both top and bottom surfaces, the top and bottom conductive tracks connecting through respective plated-through holes. The bottom side tracks extend at an oblique angle relative to the top side tracks and are spaced apart so as to provide intimate contact with the surface of the dura against which the electrode is placed during surgical procedures. The top side tracks terminate in a solder pad to which leads for the instrumentation for measuring/detecting MEPs are connected.

### BACKGROUND OF THE INVENTION

The present invention relates to an electrode for use in monitoring motor evoked potentials (MEPs) during spinal surgeries involving instrumentation. More specifically, the present invention relates to an epidural electrode that ensures proper, constant contact with the dura, thereby making it possible to continuously monitor MEPs to provide a reliable parameter for assessing nerve root compression during surgical procedures involving the spinal cord and/or spine.

Monitoring of somatosensory evoked potentials (SEPs) during surgical procedures involving the spinal cord or spine is an accepted method for reducing the incidence of neurological deficits postoperatively. However, motor deficits may occur in the absence of intraoperative SEP changes (see, for instance, Dawson, E. G., et al., 16 Spine (Suppl. 8) S361-S364 (1991); Ginsburg, H. H., et al., 63 J. Neurosurg. 296-300 (1985)) because SEPs and MEPs are conducted in different spinal cord pathways and have different blood supplies. For these reasons, methods for monitoring spinal motor pathways directly during surgical procedures have been proposed.

MEPs are elicited by activating motor pathways using transcranial electrical stimulation, transcranial magnetic stimulation, or electrical spinal cord stimulation. However, clinical application of intraoperative monitoring of MEPs has been limited by certain disadvantages and limitations, and it is therefore an object of the present invention to provide an epidural electrode for use in continuous, introperative monitoring of MEPs that addresses these disadvantages and limitations.

Various electrodes have been used for monitoring MEPs. For instance, Tamaki, T., et al. (184 Clin. Orthop. 58-64 (1984)) disclosed a “tube-type electrode, supplemented with two platinum wire coils at the end,” that they described as being “sufficiently flexible to prevent lesions of the neural tissue.” It is also known to use “strip” electrodes (described as “two 5-mm steel disks, 15 mm between centers in a silastic [sic, SILASTIC™] strip”) positioned in the epidural or subarachnoid space (Nagle, K. J., 47 Neurology 999-1004 (1996)). A review of the scientific literature reveals several other electrodes that have been used for monitoring of MEPs, but so far as is known, none of these prior electrodes are intended for and are not designed as stimulating electrodes for continuous monitoring of MEPs for assessing the integrity of spinal nerves during surgery.

It is, therefore, an object of the present invention to provide an electrode for use in continuous intraoperative monitoring of MEPs that is easily placed in intimate contact with the dura and/or in the epidural space so as to provide reliable, sensitive detection of MEPs.

There is also a need for an electrode that is unlikely to be displaced during spinal surgery, and it is also an object of the present invention to provide an electrode meeting that need.

There is also a need for an electrode that, when utilized in surgery involving the low lumbar segments, directly stimulates the nerve roots so as to lessen the effect of anesthesia on the MEPs, and it is an object of the present invention to provide an electrode for meeting that need.

Other objects, and the advantages, of the present invention will be made clear to those skilled in the art by the following detailed description of a presently preferred embodiment thereof.

### SUMMARY OF THE INVENTION

These needs are met in the present invention by providing an electrode for use in intraoperative monitoring of MEPs comprising a non-conductive, substantially rectangular, elongate strip, a conductive track on the first surface of the strip, the conductive track being in electrical contact with a lead, a conductive track on the second surface of the strip in electrical contact with the track on the first surface of the strip through a plated-through hole, the non-conductive strip being comprised of a thin, flexible material.

### BRIEF DESCRIPTION OF THE DRAWINGS

FIG. 1 is a top, plan view of a presently preferred embodiment of an electrode constructed in accordance with the teachings of the present invention.

FIG. 2 is a bottom, plan view of the electrode of FIG. 1.

### DETAILED DESCRIPTION OF THE PREFERRED EMBODIMENTS

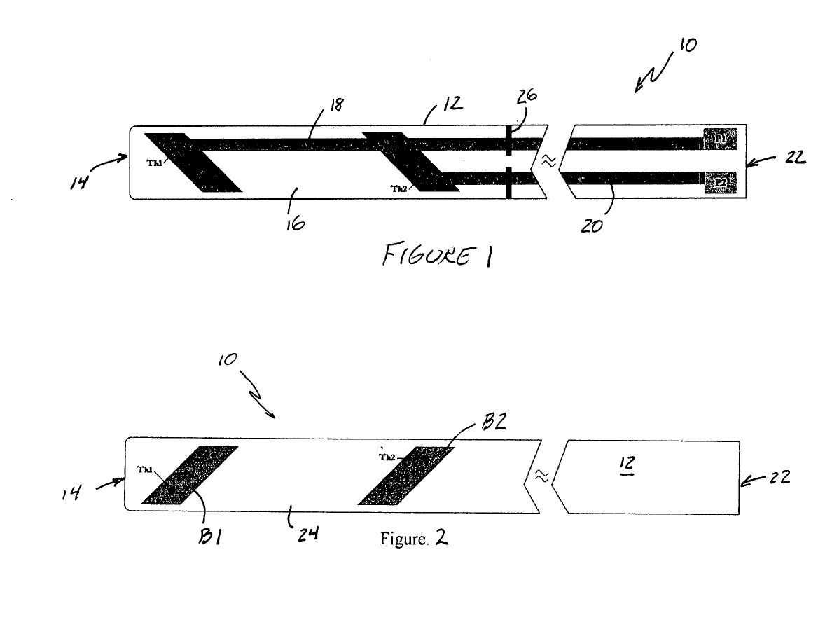

Referring now to the figures, a presently preferred embodiment of an electrode constructed in accordance with the teachings of the present invention is indicated generally at reference numeral 10 in FIG. 1. Electrode 10 is comprised of a thin (for instance, approximately 0.1-1.0 mm) flexible rectangular strip 12. Although those skilled in the art will recognize that these dimensions are not limiting, in one embodiment, the strip 12 is approximately 6-8 mm wide and 250-300 mm long, with rounded corners at one end 14. In a presently preferred embodiment, the strip 12 is comprised of a polyimide film, but those skilled in the art who have the benefit of this disclosure will recognize that other physiologically inert, highly insulative, flexible materials, or composites, or sandwiches of such materials, may also be utilized in strip 12. A list of examples of such materials includes, but is not limited to, polyimide film (trade names such as KAPTON, UPILEX, and other variants), polyester film (trade names such as MYLAR), PTFE/polytetrafluoroethylene (trade name such as TEFLON and variants), and other materials available under the trade names FR-4 (polymer/glass fiber cloth laminate) and FCCL (flexible copper clad laminate).

The top, or first, surface 16 of strip 12 is provided with tracks 18, 20 comprised of a highly conductive material. In one particularly preferred embodiment, the material comprising the tracks 18, 20 is a metal such as copper that is deposited by vapor or other thin-film deposition techniques known in the art on the first surface 16 of strip 12. Tracks 18, 20 are roughly parallel the length of strip 12, and if the strip 12 is approximately 6-7 mm in width, may be spaced approximately 1.5 mm from the edge of the strip and approximately 2 mm apart. Those skilled in the art will recognize that the dimensions and spacing set out herein are illustrative and that it is not intended that the invention be so limited. In another embodiment (not shown), strip 12 is provided with parallel stainless steel wires in place of the tracks 18, 20. The wires may be comprised, for instance, of 30 gauge wire affixed to the surface(s) of strip 12. Either way, once applied to the top surface 16 of strip 12, a layer of insulating material, such as polyimide, is applied to the top surface 16 to resist conduction between the tracks 18, 20 and the surrounding tissue when the end 14 is introduced into the epidural space in the manner described below.

The tracks 18, 20 terminate in solder pads P₁, P₂, respectively, located near the end 22 of strip 12. It is not the intention to be bound by these dimensions, but in the preferred embodiment of electrode 10 shown in the figures, the pads P₁, P₂ are both approximately 3 mm×2 mm in dimension. Solder pads P₁, P₂ are provided so that the tracks 18, 20 can be connected to respective leads (not shown) which are, in turn, connected to the instrumentation (not shown) for measuring MEPs as known in the art. When the end 14 of strip 12 is inserted into the epidural space in the manner described below, the end 22 of strip 12 extends out of the epidural space so that the conductive pads P₁, P₂ provide a point of attachment for the lead(s) of suitable instrumentation for continuous intraoperative monitoring of MEPs.

The ends of tracks 18, 20 opposite the pads P₁, P₂ terminate in plated-through holes Th1, Th2 that connect tracks 18, 20 to respective bottom side tracks B1, B2 on the bottom, or second, surface 24 of strip 12. In the embodiment shown, bottom side tracks B1, B2 are shaped as parallelograms, spaced approximately 30 mm apart, and extending at an oblique angle almost all the way across the width of the bottom surface 24 of strip 12. Again, it is not the intention to be limited to this shape, or by these dimensions, but in the preferred embodiment of electrode 10 shown in the figures, the bottom side tracks B1, B2 are both approximately 3 mm×7 mm in dimension. Although not essential, the bottom side tracks B1, B2 are angled at an oblique angle, and extend almost all the way across the second surface 24 of strip 12 so that when the strip 12 is inserted into the epidural space in the manner described below, the bottom side tracks B1, B2 extend far enough and at an angle relative to the strip so as to insure bilateral and multi-segmental stimulation of the patient's spinal column. Bottom side tracks B1, B2 are preferably comprised of a highly conductive metal such as gold and, as noted above, are in electrical contact with the tracks 18, 20 on the top surface 16 of strip 12 through the plated-through holes Th1, Th2 for the purpose of conducting current/potentials to/from the surface of the dura (not shown) with which the bottom side tracks B1, B2 are in contact when electrode 10 is properly placed for monitoring MEPs during surgical procedures involving the spine.

For the insertion of the electrode 10, the spinous process and the central lamina of the vertebra are removed at the highest vertebral level of the lumbar surgery. When the dura becomes visible, a #3 Penfield dissector or other suitable instrument is used to clear the epidural space to assist in introduction of the probe. Electrode 10 is held below the insert level (marked at reference numeral 26) and is gently introduced into the epidural space with the top surface 16 facing the surgeon. The strip 12 is inserted into the space to approximately the insert mark 26. The electrode 10 may then secured to the skin at the upper end of the surgical incision by applying sterile surgical tape.

Electrode 10 is attached to the current source from any suitable apparatus used for multimodality intraoperative monitoring. To avoid applying excessive current that could potentially injure the spinal cord, the electrode 10 is optionally be provided with current limiting circuitry (not shown) operable to limit current to, for instance, approximately 40 mA, and pulse duration to approximately 50 μsec.

The material comprising strip 12 and the minimal thickness of the strip 12 confer upon the electrode 10 of the present invention at least two advantages. First, because the material is flexible and the strip is so thin, electrode 10 is highly flexible, facilitating proper placement of the electrode in intimate contact with the dura. Second, the surface tension created by the fluids present in the epidural environment in which electrode 10 is placed is such that the electrode is held in intimate contact with the surface of the dura, thereby insuring intimate contact with the dura, and consequently, both good conductivity and a substantial decrease in the likelihood of displacement of electrode 10 during surgical procedures.

The increased conductivity of the electrode of the present invention has been demonstrated by comparing baseline and intra-surgical threshold/absence of MEP. Basal MEP findings in 14 patients, in terms of increased threshold/absence of MEP, correlated with clinical findings of radiculopathy in 12 patients and with radiological findings in all 14 patients. During surgery, after bilateral decompression, the MEP threshold improved in all patients, unilaterally in 6 patients and bilaterally in 8 patients. In 3 patients, SEP deteriorated during surgery while MEP showed improvement.

Although described in terms of the embodiments shown in the figure, the embodiment is shown to exemplify the present invention and not to limit the scope of the invention, it being recognized by those skilled in the art that certain changes can be made to the specific structure of the embodiment shown and described without departing from the spirit of the present invention. In the case of one such change described above, the tracks 18, 20 are replaced by stainless steel wire. Another modification that is intended to fall within the scope of the present inventions is to make the strip 12 in the form of a laminate, with layers of conductive material substituting for tracks 18, 20 sandwiched between non-conductive, insulative layers. Similarly, a material other than the material comprising the strip 12 may be utilized as the layer of insulating material covering the tracks 18, 20 on the first surface 16 of strip 12, the different material being selected because it confers additional desirable properties to the strip 12, such as, for instance, a decreased coefficient of friction when wetted so as to assist in the insertion of strip 12 into the epidural space. Another modification relates to improvements in the strip for the purpose of placement and retrieval of electrode for surgical procedures, it being understood by those skilled in the art that the strip 12 may be provided with, for instance, a hole or other structure at each by which the strip may be grasped for ease of manipulation. All such modifications, and other modifications that do not depart from the spirit of the present invention, are intended to fall within the scope of the following claims.

What is claimed is:

1. An electrode for use in monitoring motor evoked potentials during spinal surgery comprising:
an elongate strip;
a conductive track on the first surface of said strip, the conductive track being adapted for electrical connection with a lead;
a conductive track on the second surface of said strip in electrical contact with the track on the top surface of said strip through a plated-through hole; and
said strip being comprised of a thin, flexible, electrically non-conductive material.

2. The electrode of claim 1 wherein said strip is comprised of a polyimide film.

3. The electrode of claim 2 wherein the conductive track on the first surface of said strip is deposited on said strip.

4. The electrode of claim 1 wherein the conductive track on the first surface of said strip is comprised of copper and the conductive track on the second surface of said strip is comprised of gold.

5. The electrode of claim 1 wherein the conductive track on the second surface of said strip is angled relative to the conductive track on the first surface of said strip.

6. The electrode of claim 1 wherein the first surface of said strip is provided with a second conductive track extending further along the length of said strip than the first conductive track and connecting through a second plated-through hole to a respective second conductive track on the second side of said strip, the second conductive track on the second side of said strip being spaced apart from the first conductive track on the second side of said strip.
